(19) United States
(12) Patent Application Publication
(10) Pub. No.: US 2010/0089406 A1
(43) Pub. Date: Apr. 15, 2010

(54) Method and device for male contraception

(76) Inventor: Elena Kachiguina, San Carlos, CA (US)

Correspondence Address: Elena Kachiguina, 6 Azalea Lane, San Carlos, CA, 94070, US

(21) Appl. No.: 12/287,732

(22) Filed: Oct. 14, 2008

(51) Int. Cl. A61F 6/02

(52) U.S. Cl. 128/842

(57) ABSTRACT

The present invention relates to a method and device for male contraception. In one aspect, the method and device provide temporal and reversible infertility of mammal males without suppressing spermatogenesis. In an aspect, a method of contraception for use with reproductive cells that pass through a tube in a body is described in which energy is applied through the tube wall to some of the reproductive cells that pass through the tube to reduce fertilization ability of the some of the reproductive cells. In another aspect, the device is adapted for use with reproductive cells that pass through a tube in a body, and includes an energy transmitter that delivers energy to the reproductive cells that pass through the tube in the body.

### PRIORITY CLAIMS

This application claims priority to: U.S. provisional application No. 60/791,586 filed Apr. 13, 2006 entitled “Method and Device for Providing Temporal Infertility”; U.S. provisional application No. 60/801,495 filed May 18, 2006 entitled “Method and Device for Mammal Male Contraception”; U.S. provisional application No. 60/849,316 filed Oct. 3, 2006 entitled “Method and Device for Male Contraception”; and U.S. provisional application No. 60/855,318 filed Oct. 30, 2006 entitled “Method and Device for Male Contraception”, each and all of each are incorporated by reference herein.

### FIELD OF THE INVENTION

The present invention relates to a method and device for male contraception. In particular, the subject of the invention is a method and device for providing temporal and reversible infertility of mammal males without suppressing spermatogenesis.

### BACKGROUND OF THE INVENTION

Conventional male contraceptive methods are essentially of two types.

One type, condoms, traps sperm that is otherwise viable. Usage of condoms has many known deficiencies, particularly with respect to the possibility of defects in their construction and/or usage that can lead to their ineffectiveness.

The other type, a vasectomy, permanently prevents the ejaculation of sperm, typically by cutting the vas deferens. While effective at preventing pregnancy, the fact that a vasectomy is permanent is undesirable to many people.

### SUMMARY OF THE INVENTION

The present invention relates to a method and device for male contraception.

In one aspect, the method and device provide temporal and reversible infertility of mammal males without suppressing spermatogenesis.

In an aspect, a method of contraception for use with reproductive cells that pass through a tube wall of a tube in a body is described in which energy is applied through the tube wall to some of the reproductive cells that pass through the tube to reduce fertilization ability of the some of the reproductive cells.

In a particular aspect, the method is provided by applying energy pulses to the vas deferens to reduce fertilization ability of at least some of the sperm that travels through the vas deferens.

In another aspect, the device is adapted for use with reproductive cells that pass through a tube wall of a tube in a body, and includes an energy transmitter that delivers energy to the reproductive cells that pass through the tube in the body.

In a particular aspect, the device is a miniature implantable ultrasonic transducer positioned on the vas deferens inside the scrotum, which transducer produces pressure pulses that reduce fertilization ability of at least some of the sperm that travels through the vas deferens.

In another aspect, the method comprises positioning an ultrasound transducer on the vas deferens and applying ultrasound energy primarily to the internal volume of the vas deferens.

### BRIEF DESCRIPTION OF THE DRAWINGS

These and other aspects and features of the present invention will become apparent to those of ordinary skill in the art upon review of the following description of specific embodiments of the invention in conjunction with the accompanying figures, wherein:

FIG. 1 shows a cross-section of the implantable device according to the present invention.

FIG. 2 shows a cross-section of the vas deferens and device according to the present invention positioned over the skin of the scrotum.

FIG. 3 shows a cross-section of the device according to another embodiment of the invention placed on the vas deferens.

FIG. 4 shows cross-section of the vas deferens and device of FIG. 3 along the longitudinal axis of the vas deferens.

FIG. 5 shows a cross-section of the vas deferens and device according to another embodiment of the invention.

FIG. 6 shows a cross-section of vas deferens and device of FIG. 5 along the longitudinal axis of the vas deferens.

FIGS. 7(a)-(e) shows the implantable device installation procedure according to the present invention.

FIGS. 8(a)-(d) show a device according to the present invention and exploded views of one embodiment of the device design with a flexible piezoelement.

FIGS. 9(a)-(d) show a device according to the present invention and exploded views of another embodiment of the device design with rigid piezoelements.

FIG. 10 illustrates another device embodiment according to the present invention with an additional actuator.

FIGS. 11 (a)-(c) illustrate another device embodiment according to the present invention.

FIG. 12 shows the cross-section of the device according to another embodiment of the present invention.

FIGS. 13 (a)-(b) illustrate another embodiment of the device according to the present invention that is provided with an elastic element that is coaxial to the emitters.

FIGS. 14(a)-(b) show block diagrams of an electrical and control schematic for the device according to the present invention.

### DETAILED DESCRIPTION OF THE PREFERRED EMBODIMENTS

The following overview is first provided. A further description with reference to the drawings follows thereafter. The descriptions in the overview and with reference to the drawings are intended to highlight various different aspects and features of the present invention.

The method comprises positioning an energy transmitter, such as an ultrasound transducer, on the vas deferens and application of energy, such as ultrasound energy, electrical energy, electromagnetic energy, or light energy, primarily to the internal volume of the vas deferens. Testis and epididymis are not subject to ultrasound energy.

Though a preferred embodiment described below discusses the invention in terms of ultrasound energy, electromagnetic energy and light energy can be used, with the electromagnetic components of the electrical, electromagnetic or light signals, and the polarization components of the light signal, conforming to the properties of the reproductive cell, and in particular the cell membrane of the sperm, in order to reduce the fertilization ability to the greatest extent using the lowest amount of energy.

The method comprises, in a preferred embodiment that will be described in detail below, positioning a miniature implantable ultrasonic transducer on the vas deferens inside the scrotum.

The method, according to another embodiment, comprises pinching the scrotum of the male and capturing the vas deferens in the pinched region, positioning a wearable ultrasonic transducer on the scrotum skin in the pinched region and attaching it to the skin, applying energy through the skin covering the pinched region.

The pinching and device attachment to the skin may include skin piercing.

The ultrasound energy power is set to be sufficient to damage spermatozoa tail for at least 70% of sperm.

The ultrasonic energy is set to be sufficient for temporal increase of temperature of the sperm volume inside the vas deferens.

The ultrasound energy and frequency is set to modify conditions of sperm flow or chemical reactions inside the vas deferens that results in reduction of spermatozoa motility or fertility quality of sperm.

The ultrasound energy is applied by pulses. The pause between the pulses is less than the time of sperm travel through the zone of ultrasound energy application.

The ultrasound energy is applied from several directions with intersection in the center of the vas deferens.

The ultrasound wave length is less than the cross-section size of the vas deferens. In a preferred embodiment the ultrasound frequency is set to be close to the resonance frequency of the spermatozoa or its parts and components.

The device for temporal and reversible infertility comprises a miniature implantable ultrasound transducer, an energy source and electronic circuitry for control of the transducer. The device positioned on the external surface of the vas deferens and secured to it. The ultrasound transducer emitting surface is directed toward the internal volume of the vas deferens.

The device may comprise an ultrasound reflector positioned symmetrically to the ultrasound transducer relative the vas deferens tube axis.

The space between the ultrasound transducer and reflector and external surface of the vas deferens is filled with a filling substance that has ultrasound impedance value close to the same of the tissue of the vas deferens.

The device may comprise several ultrasound transducers positioned along an arc that is concentric to the vas deferens tube. The ultrasound transducers are directed toward the axis of the vas deferens tube.

The device for temporal and reversible infertility comprises a fixture for pinching a scrotum skin with the vas deferens inside, a wearable ultrasound transducer, an energy source and electronic circuitry for control of the transducer. The device positioned on the scrotum skin in the pinched area and secured to it. The ultrasound transducer emitting surface is directed toward the axis of the vas deferens tube.

In one embodiment, the scrotum skin is pinched with a “permanent” pin (a rivet, fastener) pierced through the scrotum skin. Several pins may be placed this way along the pinched vas deferens. The device is attached and secured to the pin(s). The ultrasound transducer is directed toward the axis of the vas deferens. The device is removable. The space between the scrotum skin and ultrasound transducer is filled with a filling substance with high water vapor and oxygen transmission rate.

Any suitable power source (physical, chemical or mechanical such as battery, capacitor, accumulator, generator, energy generating cell, vibration energy or heat scavenging generator, etc.) may be used for powering the ultrasound transducer and control circuitry.

In a preferred embodiment the power source is a combination of a battery, capacitor or rechargeable battery, and thermal or vibration electrical generator.

The device may be provided with rechargeable battery or accumulator. The battery is periodically charged by an external power supply through electrical contacts or by electro and/or magnetic alternating field. The rechargeable circuitry is provided with an antenna for generating induced electro-energy due to external electro and/or magnetic field.

The device may be provided with a miniature power generator that scavenges the energy from the ambient vibrations, temperature variations or electromagnetic fields.

The control circuitry is responsible for at least one of the following actions:

generate the necessary voltage, frequency and waveform for driving the ultrasound transducer;

control duration and duty cycle of pulse operation of the transducer;

control battery charge and alarm in case of the device malfunction or low battery power;

generate radio wave or audible signal to inform the user about problems;

keep the record of device operation and alarms;

communicates with an optional external remote control for the device turn on and off and the operation record report per user's demand.

The device is fabricated using known technologies employed for manufacturing micro-electro mechanical systems. The ultrasound transducer may be fabricated from piezoceramic or solid state material (e.g. silicon capacitive ultrasound transducer). The circuitry and power source may be integrated with the ultrasound transducer in an enclosure and sealed by bio-compatible material. The material may be a metal (titanium, stainless still), or ceramic, or polymer (parilene, polyethylene, silicone).

The ultrasound emitter may be acoustically isolated from a surrounding tissue and other device components by:

Using a layer of material with higher acoustical impedance than the acoustic emitter material impedance so that the acoustical waves are reflected from the boundary of the high acoustical impedance material;

Using a cavity filled with a gas or porous material to minimize coupling to the surrounding tissue. The cavity is hermetically sealed and:

i. The cavity walls may have flexible elements to compensate variations in the ambient pressure by contraction or expanding the cavity volume.

ii. The gas in the cavity may be at higher pressure than ambient pressure. That prevents potential collapse of the cavity walls due to external mechanical stress and resulting coupling to the tissue.

iii. The cavity walls may be made rigid. In that case the cavity may be held under reduced pressure or vacuum to further minimize acoustic coupling to the surrounding tissue.

The ultrasound transducer frequency may be chosen from the range 50 kHz-150 MHz. The ultrasound emitting power is in the range 0.001-1000 W/sq.cm. The optimal ultrasound frequency and power that are sufficient for spermatozoa motility reduction may be easily defined by a person skilled in the field after a few routine experiments for a given mammal type and the physical design of the ultrasound transducer and its attachment.

In one embodiment, the width of the ultrasound transducer is less than the cross-section size of the lumen. In some embodiment the width of the ultrasound transducer is in the range of 0.05 to 10 mm. In some embodiments the emitter is provided with a lens or optical elements to reduce the beam width along the lumen axis. Several ultrasound transducers may be placed along the vas deferens. The ultrasound transducers operate synchronically or with time phase shift.

More than one ultrasound transducers may be placed around the vas deferens so that the irradiated beams would intersect at, predominantly, the same point at the longitudinal axis of the vas deferens. The ultrasound transducers may operate synchronically. That allows to achieve maximum intensity of the ultrasound in the vas deferens lumen, and, at the same time, to minimize intensity of ultrasound in the vas deferens smooth muscle.

The frequency of the ultrasound transducers operation and the distance from the emitting surface of the ultrasound transducer and the opposite reflecting surface may be chosen so that distance is equal to integer number of the half of the ultrasound wave length. That creates a standing ultrasound wave between the ultrasound transducer and the reflecting surface with the antinode at the longitudinal axis of the vas deferens. That provides maximum pressure and stress in the lumen.

The ultrasound transducer is on continuously or in a pulse mode to save the energy and avoid thermal heating of the tissue. The pause duration between the pulses is chosen less then the time of sperm travel from proximal to distal ends of the ultrasound irradiated zone within the device.

The device may be provided with at least one sensor that detects peristaltic movement of the vas deferens. The sensor type may be pressure, stress or displacement based. The sensor is facing the vas deferens external surface and senses the vas deferens external surface movement due to peristaltic contractions. The ultrasound transducer pulse rate is changed per the rate of the vas deferens peristaltic contraction: the higher the peristaltic contraction rate the higher ultrasound pulse rate. That allows reduction of power consumption of the ultrasound transducer and extends the power source life.

Two sensors are used also for detection of the direction of the peristaltic movement of the vas deferens. The sensors are positioned along the vas deferens. They may be integrated into the device. The capacitive ultrasound transducers may be used as the pressure sensors between the ultrasound pulses.

The ultrasound pulse rate is increased only when the vas deferens peristaltic movement is directed from testis.

The device may be provided with an actuator for temporal pinching the vas deferens and creating temporal constrains for sperm flow

The device is provided with the electrodes for forming an electrical field that is applied to the internal volume of the vas deferens. The electrodes are isolated to minimize electrical current. The electrical current is less than 1 mA. The minimal electrical field strength is 0.001 V/cm. The maximum electrical field strength is limited by the electrical breakdown of the electrode isolation.

The spermatozoa carry negative electrical charge distributed over the tail area. The spermatozoa are moved by the electrical field toward the anode. The electrical field causes orientation of the spermatozoa so that it moves toward the anode with its tail leading the head. The higher the electrical filed the stronger the effect on spermatozoa.

In one embodiment the anode is placed near the device end facing the testes, and the cathode is placed near the other end. The electrodes may be made in form of rings positioned around the vas deferens and electrically isolated from the surrounding tissue. The electrical field is directed along the vas deferens. The force between the spermatozoa charge and the electrical field is pulling the spermatozoa toward the anode against the natural peristaltic movement of the sperm. That creates a mechanical stress that may damage the spermatozoa tail and reduce sperm fertility. Also the spermatozoa stay longer within the device ultrasound exposure area that reduces possibility of mistreated spermatozoa.

In another embodiment the electrodes are located opposite to each other in the same cross-section of the vas deferens. The electrical field pulls the spermatozoa toward the lumen wall at the anode side. The tail is leading so it hits the lumen wall first followed by the spermatozoa head. That causes stress and damage to spermatozoa tail resulting in sperm motility loss. When polarity of the electrodes is changed to opposite, the spermatozoa are pulled toward the new anode that was the cathode at initial polarity. The spermatozoa are turned around by the electrical field and moves to the opposite wall by tail again. The electrical field may have alternating polarity. In that case every spermatozoa change orientation many times while transported along the device by the vas deferens peristaltic.

The electrodes and ultrasound transducers may be combined in one device.

In another embodiment the device is provided with a watchdog control circuitry that measures charge status of the power source. If the power source charge is below a predetermined level due to malfunction or discharging, the watchdog control circuitry generates a signal to the ultrasound transducer control circuitry to vibrate at a frequency below 22 kHz. That vibration is not audible yet but may be sensed by the user body. It provides the user signal that the power source has to be charged. In another embodiment the device is provided with a light source, such as light emitting diode, to indicate the status of the device or the battery.

The watchdog control circuitry may be provided with an antenna so that to send a radio signal to a remote receiver. The signal is coded to distinct a signal that confirms a normal operation of the device and power source from the signal that indicates a problem or low charge status. The antenna may be used for receiving a wake-up signal from the remote receiver and additional power for sending the response. That allows to save the charge of the power source.

In another embodiment, the device may be provided with a watchdog sensor that senses the ultrasound pulses. The watchdog sensor is used by the watchdog control circuitry to confirm that ultrasound generation operates normally.

In some embodiments the ultrasound transducer is kept on for a long time to achieve temperature rise of the internal volume of the vas deferens.

A discussion of the invention with reference to the drawings is provided hereinafter.

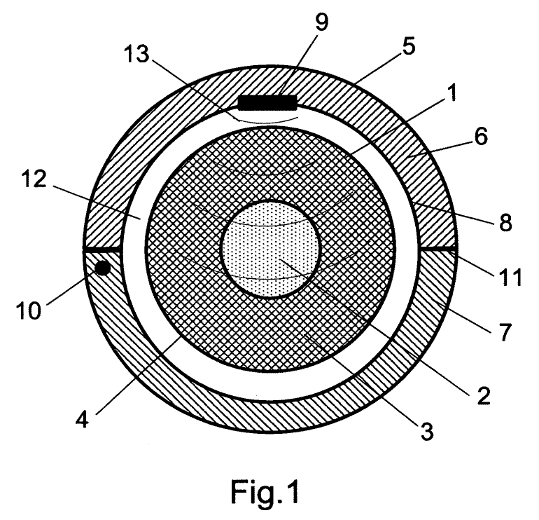

FIG. 1 shows a cross-section of the implantable device positioned at the vas deferens inside the scrotum. The vas deferens 1 has a lumen 2 with diameter about 0.25-0.56 mm that contains sperm. The internal volume 2 is surrounded by smooth muscles 3 that form the vas deferens tube with external surface 4 with diameter about 2.2-4 mm.

The device 5 is clipped-on the vas deferens 1. The device 5 is shown as a cylinder comprising two half cylinder parts 6 and 7. The internal diameter 8 of the device 5 is slightly larger (0.5-3 mm) than the external diameter 4 of the vas deferens 1 to preferably avoid mechanical compression of the vas deferens. The length of the cylinder is typically about 1-12 mm.

In shown embodiment, the half cylinder 6 is provided with an ultrasonic transducer 9 that emits ultrasonic energy 13 toward the internal volume 2 of the vas deferens 1. The second half cylinder 7 is used for clamping of the first half cylinder around the vas deferens 1. The second half cylinder 7 may be provided with hinge 10 and lock 11.

The control circuitry and power source (not shown) may be integrated in either half.

The gap 12 between the external surface 4 of the vas deferens 1 and internal surface of the ultrasound transducer 9 is filled with biocompatible compliant gel that fills voids between the vas deferens and the internal surface of the device. The gel provides a continuous path for ultrasound waves 13 for any particular vas deferens size. The gel has ultrasound impedance that is close to the vas deferens smooth muscle impedance.

The cylinder second half 7 may be utilized as a reflector for the ultrasonic waves 13 to redirect non-absorbed ultrasonic energy back to the internal volume 2. In some embodiments, the distance between the emitting surface of the ultrasound transducer 9 and the reflecting surface of the second half cylinder 7 is set to be approximately equal to a half wave length of ultrasound in the vas deferens smooth muscle tissue. This distance L is calculated as: L=Vsm/(2*Fus), where Vsm is the sound velocity in the smooth muscle tissue (˜1550 m/s), and Fus is the ultrasound frequency. The human vas deferens diameter is varied from 2.2 to 4 mm. The distance L of the device is chosen 4 mm to cover the range of vas deferens size variation. In some embodiments, the device size may be adjusted or chosen from the set of different sizes to accommodate a particular size of the vas deferens. The ultrasound frequency Fus has to be Fus=Vsm/(2*L) to satisfy the standing wave requirement. If L=4 mm then Fus is 194 kHz.

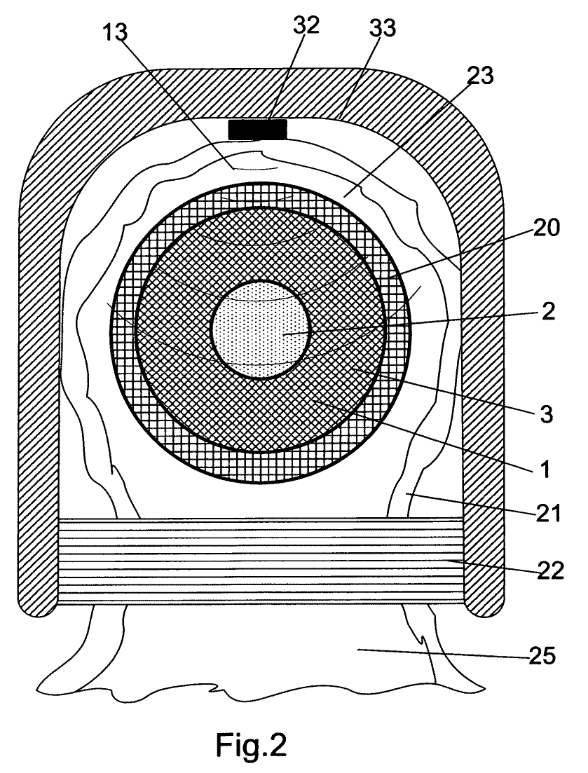

FIG. 2 shows a cross-section of the vas deferens 1 and device 31 positioned over the skin 21 of the scrotum 25.

The vas deferens 1 together with the connecting issue and vas sheath 20 is pinched by the scrotum skin 21. An optional pin 22 may be pierced through the skin 21 to keep the shape of the pinched skin 21 and be used for mounting the device 31. The device 31 comprises an ultrasound transducer 32. The device 31 is mounted over the pinched skin 21 so that the ultrasound transducer 32 would be located near the pinched skin 21 and directed toward the internal volume 2 of the vas deferens 1. The gap between the ultrasound transducer 32 and scrotum skin 21 is filled with biocompatible and pliable gel. The ultrasound transducer 32 emits ultrasonic waves through the gel, scrotum skin 21, connecting tissue 23, vas sheath 20, and smooth muscles 3. The pin 22 may be used as a reflector for the ultrasonic waves 13 to redirect non-absorbed ultrasonic energy back to the internal volume 2. More than one ultrasound transducers may be placed along the internal surface 33 of the device.

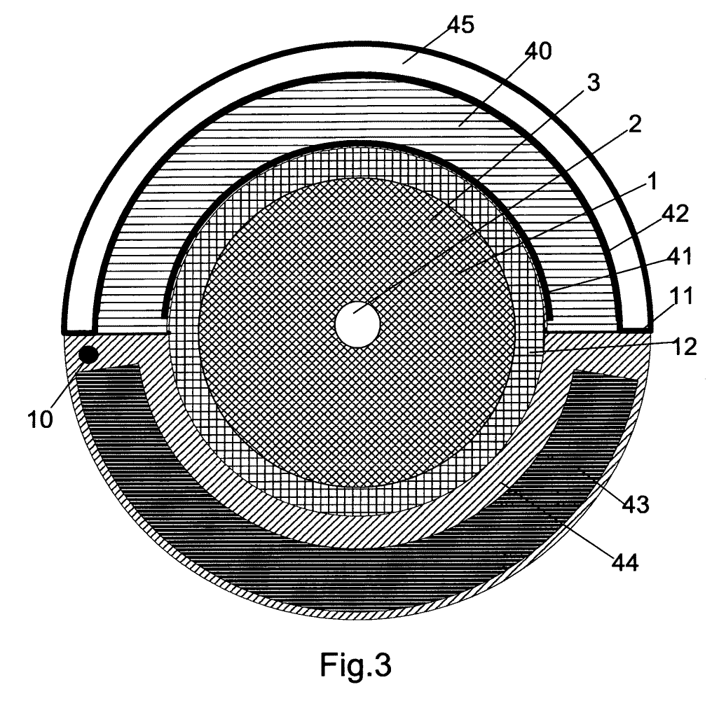

FIG. 3 shows a cross-section of the device per another embodiment placed on the vas deferens 1. The upper half-cylinder part of the device consists of a piezoelectric element 40, made in shape of a circular segment. The internal radius of the segment is larger than the vas deferens 1 radius. The piezoelectric element 40 is provided with metal contact 41 on the internal surface, and a metal contact 42 on the external surface. The piezoelectric element 40 is placed concentrically to the vas deferens 1. The gel 12 is used to fill voids between the piezoelectric element 40 and vas deferens 1. When voltage is applied to the electrodes 41, 42 the piezoelectric element 40 contracts radially causing pressure wave toward the center of the lumen 2. The lower half cylinder is attached to the upper half cylinder with the hinge 11 and lock 12 to clip the parts around the vas deferens. The lower part may have integrated battery and serve as a reflector 44. The external part of the piezoelectric element is provided with acoustically isolating member 45 from the surrounding tissue by: a) a layer of a material with high impedance; or b) a hermetically sealed cavity filled with a gas or porous media

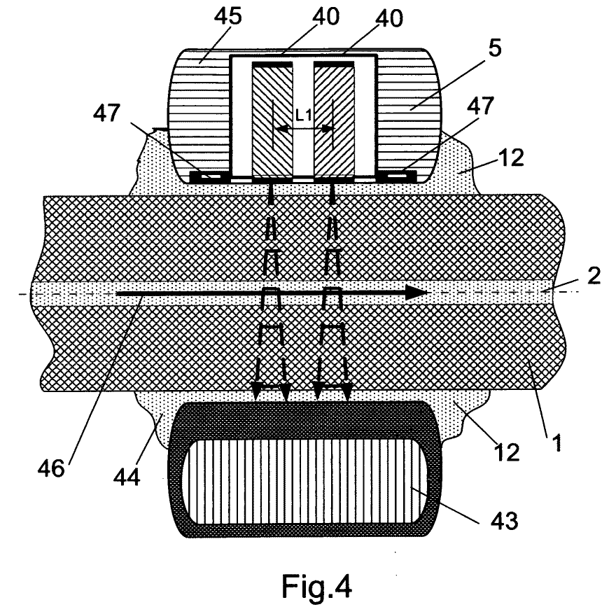

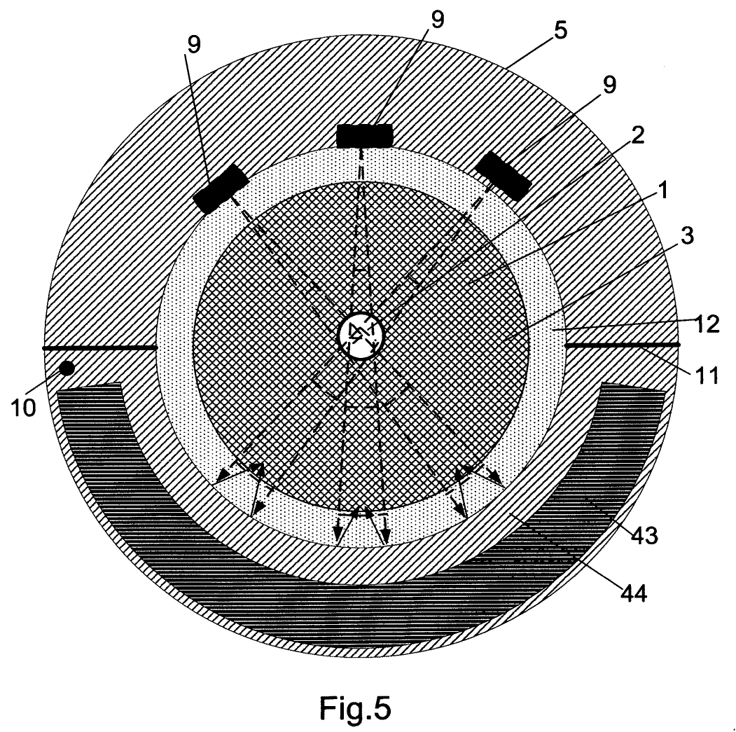

FIG. 4 shows cross-section of the vas deferens and device of FIG. 3 along the longitudinal axis of the vas deferens. The device may be provided for illustration with two or more piezoelectric segment elements 40. In one embodiment, the distance L1 between the ultrasound transducer centers is set to be approximately equal to a half wave length of the sound in the lumen fluid, that is calculated as L1=V1f/(2*Fus), where V1f is the sound speed in the lumen fluid (˜1400 m/s). Such placement of the ultrasound transducers provides resonant superposition of the longitudinal hydraulic shock waves in the lumen created by the acoustic pressure pulse of every ultrasound transducer. For Fus=194 kHz, the distance L1=3.6 mm. The hydraulic shock wave create additional stress waves in the lumen fluid 46 that may cause damage to the suspended spermatozoa and result in reduced fertility. The pressure sensors 47 are installed at the internal surface of the upper half 5 to detect peristaltic movement of the vas deferens 1.

In other embodiment, the ultrasound transducers are located at other distances from each other. The operation of the ultrasound transducers may be performed with a time phase shift from each other. The transducers may be positioned symmetrically or asymmetrically relative to the axis of the lumen.

FIG. 5 shows a cross-section of the vas deferens 1 and device per another embodiment. The upper half 5 of the device is provided with several acoustic emitters 9. The emitters are directed toward the center of the lumen 2 and their surfaces are tangential to an arc with the center at the longitudinal axis of the lumen 2. Ultrasound beams, generated by the emitters 9, intersect in the lumen 2 and create higher pressure in the lumen 2 than in the vas deferens's smooth muscle 3.

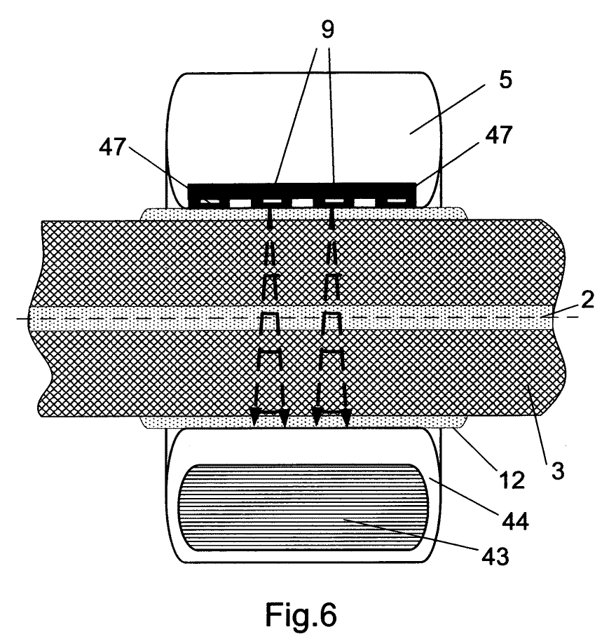

FIG. 6 shows a cross-section of vas deferens and device of FIG. 5 along the longitudinal axis of the vas deferens. Optional pressure sensors 47 are shown.

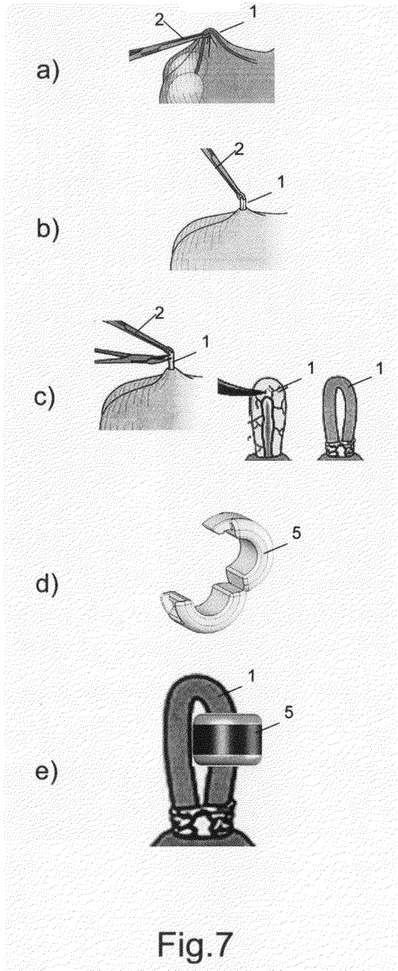

FIGS. 7(a-e) show the implantable device installation procedure.

Once the area is frozen, the doctor locates the vas deferens 1 one at a time, FIG. 7(a). He gently holds each vas deferens 1 between his fingertips, and grasps them with a specially designed ringed clamp 2. With a pair of special forceps, he makes one tiny puncture (approximately 4-10 millimeters long) into the skin on the scrotum. The vas can then be delivered through the incision FIG. 7(b), along with several layers of overlying tissue, including the vas sheath. A longitudinal incision is made through these tissues until the typically shiny white tube is seen. Once the vas lies within the grasped tissue, a towel clamp is placed around the vas sheath. The vas is grasped with toothed forceps and lifted up out of the sheath. Using a curved mosquito clamp, the connective tissue and the vas artery are separated from the tube itself, FIG. 7(c).

Once the vas deferens have been brought out through the opening in the scrotum, doctor applies an optional gel to the internal surface of the device FIG. 7(d) and attaches (clip on) the device to the vas tube, FIG. 7(e). The doctor then gently places the vas tube with attached device back in the scrotum. The same procedure is done through the same small opening on the other sperm tube. The opening is finally closed with a suture or surgical glue or covered with a gauze pad.

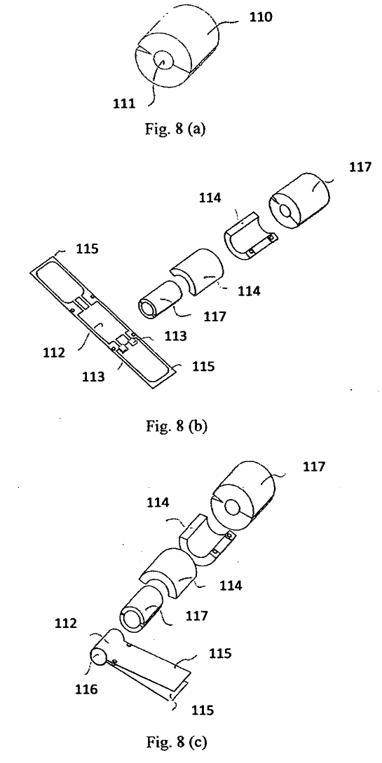

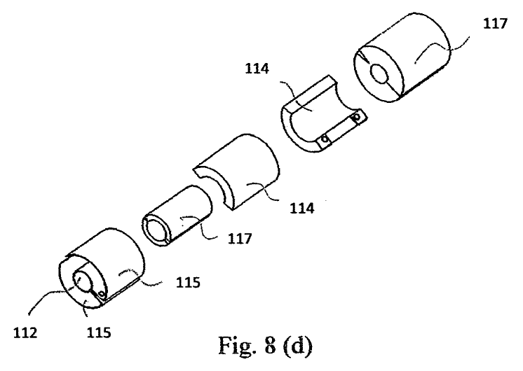

FIG. 8(a-d) show a device and exploded view of a one embodiment of device design with flexible emitter. The device 110 (FIG. 8(a)) is made in form of a cylinder with axial opening 111 for the vas deferens. A flexible emitter 112 (FIG. 8 b-d) is provided with patterned thin film metallization for the electrodes, contacts to a control and power circuitries 113 (made as integrated circuitries on a chips), contacts to batteries 114 and optional antennas 115 for external RF battery charging or communication. The polymer polyvinylidene fluoride or any other flexible piezoelectric material or composite may be used. The control and power chips 113 are attached to the thin metal circuitry by soldering or by use of a conductive polymer.

The flexible piezoelectric 112 is rolled to form a cylindrical cavity 116 with an internal diameter larger that the vas deferens diameter. An optional backing element 117 with is placed around the piezoelement 112 to keep ultrasound emission inside the cavity 116. The batteries 114 are placed around the cavity 116 and backing 114. The antennas 115 (FIG. 8d) are formed in cylindrical shape to overlap the batteries 114. Finally the assembly is encapsulated in biocompatible plastic or resin, e.g. silicone, 117.

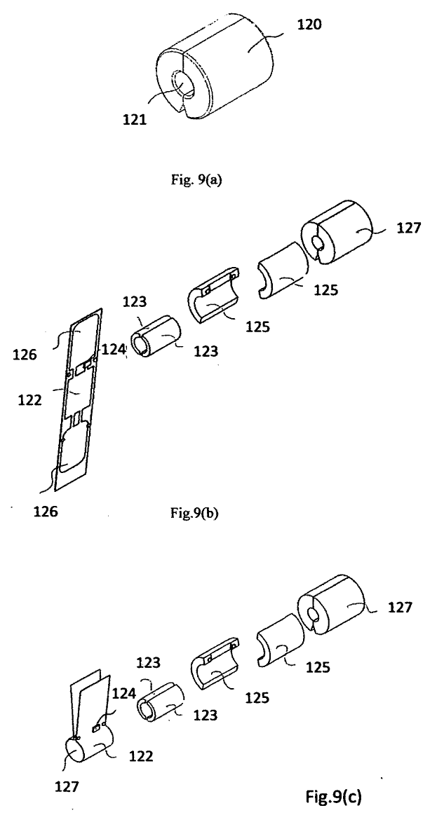

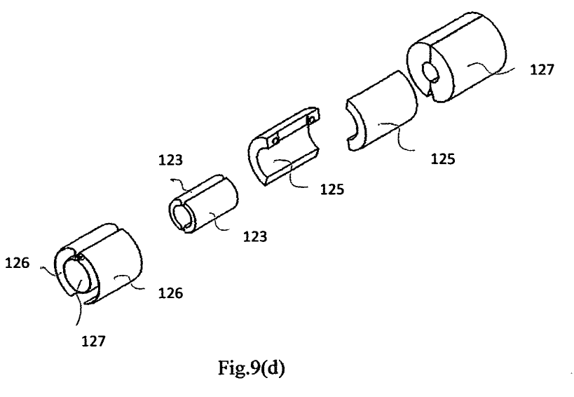

FIGS. 9(a-d) show a device and exploded view of a one embodiment of device design with rigid emitter or piezoelements. The rigid emitters 123 may be made of piezoelectric ceramic or quartz or composites. The elements 120 are made in form of semicylinders with the internal diameter larger than the vas deferens diameter. The device 120 (FIG. 9(a)) is made in form of a cylinder with axial opening 121 for the vas deferens. A flexible circuitry 122 (FIG. 8 b-d) is provided with patterned thin film metallization for the electrodes to piezoelements 123, contacts to a control and power circuitries 124 (made as integrated circuitries on a chips), contacts to batteries 125 and optional antennas 126 for external RF battery charging or communication. The polymer polyimide or any other flexible materials or composite may be used. The control and power chips 124 are attached to the thin metal circuitry by soldering or by use of a conductive polymer.

The flexible circuitry 122 is rolled to form a cylindrical cavity 127 with an internal diameter larger that the sum of the vas deferens diameter and rigid piezoelement 123 thickness. The piezoelements 123 are placed inside the cavity 127, coaxial to its axis and attached to its walls. The batteries 125 are placed concentrically outside the cavity 127 and the piezoelements 123. The antennas 126 (FIG. 9d) are formed in cylindrical shape to overlap the batteries 125. Finally the assembly is encapsulated in biocompatible plastic or resin, e.g. silicone, 127.

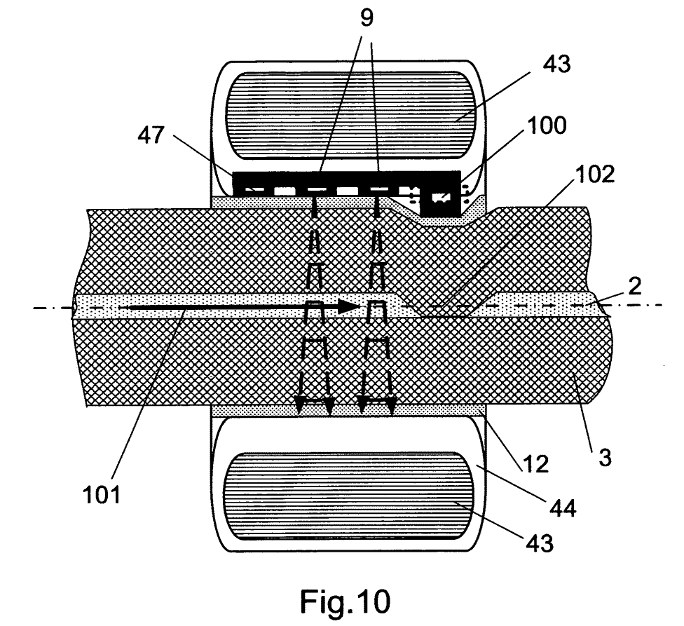

FIG. 10 shows another device embodiment with an additional actuator. The device may be additionally provided with an actuator 100 that is used for temporal deforming and pinching 102 the vas deferens 3. In the result of pinching, the lumen 2 is at least partially closed and the sperm flow stops. That may be used to prevent high sperm flow rate through the device due to increased peristaltic activity of the vas deferens right before or during ejaculation. A sensor 47 is used to detect the beginning and end of increased vas deferens peristaltic activity, and corresponding moments of turning the actuator on and off.

The mechanical pinching of the vas deferens may be used also for mechanical damage of spermatozoa

The actuator 100 may be done on base of known techniques of mechanical, electrical, thermal or magnetic mechanisms or expansion of material. The preferred embodiment is piezoactuator that expands under applied electrical potential thus pinching the vas deferens, and retracts after removal of the potential.

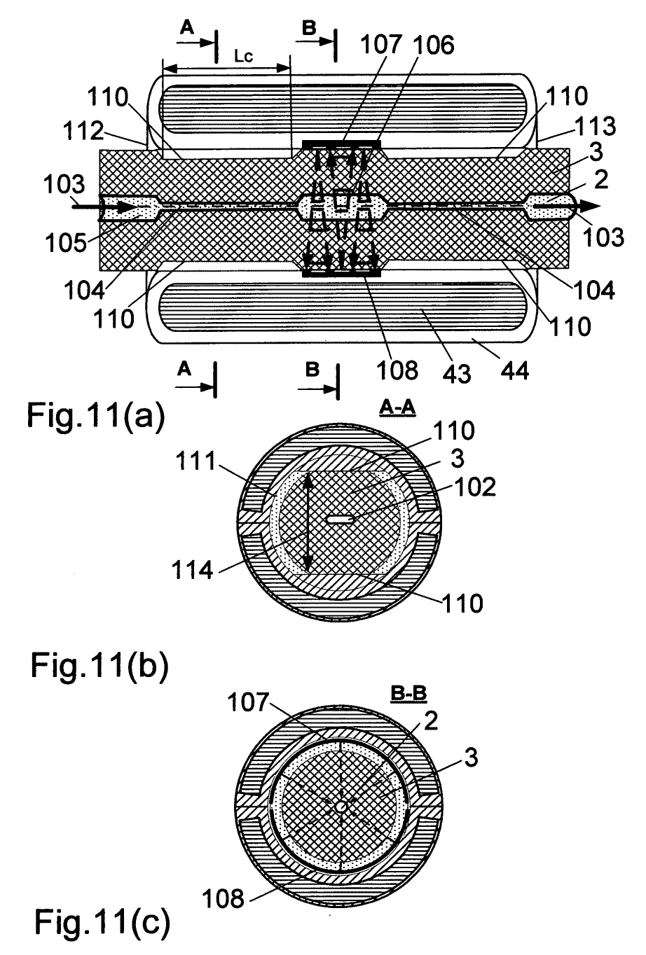

FIG. 11 (a) illustrates another device embodiment. FIG. 111(b) shows cross-section A-A at the FIG. 11(a). FIG. 11(c) shows cross-section B-B at the FIG. 11(a). In this embodiment the device is provided with at least one clip or internal passage narrowing 110 that reduces the vas deference 3 cross-section size without occlusion. The clip 110 may be made by reducing internal diameter 111 of the device (FIG. 11(b)). The clips 110 are located at both upstream 112 and downstream 113 device sides along the vas deferens 3. The clip size is made to provide deformation on the vas deferens 3 along one diameter 114 to reduce size of the lumen 2 opening. The cross-section deformation of the vas deferens 3 in the range of 0.1-0.5 mm on the radius results in collapse of initially round lumen cross-section 2 with diameter ˜0.2 mm to narrow slit 102 (FIG. 11(b)) with approximate dimensions 0.02×0.3 mm. Such small vas deferens deformation does not cause occlusion of the blood flow in the vas deferens smooth muscle and slow flow 103 of the sperm due to pressure build up from the epididimus. However, this pinching and lumen volume reduction results in termination of fast sperm flow that may be caused by the peristaltic contraction of vas deferens smooth muscle. The longitudinal size Lc (FIG. 11(a)) of the clips or device size lessening is made more than vas deferens diameter.

In other embodiments, several pinch regions 104 may be made along the longitudinal axis 105 of the device. Zones 106 (FIGS. 11 (a,c)) of non-deformed vas deferens 2 between the pinch regions 104 are used for the pressure pulse treatment of the slow moving sperm by the emitters 107 and 108.

In some embodiments, the device internal cross-section size 111 at the zone 106 where the ultrasound emitters 110 are located may be made also with the dimension that is less than the vas deferens 3 diameter.

The device according to the present invention may also be used in conjunction with other means of reducing male fertility in order to reduce negative effect of ultrasound on intravasal tissues, particularly on epithelial cells of vas lumen and smooth muscles of the vas deferens. For example use of reduced doses of fertility reducing hormonal and other drugs such as androgens and hormone combinations—testosterone alone or testosterone/progestin/gonadotropin-releasing hormone (GnRH) antagonists and agonists/Progesterone and synthetic gestagens/estrogens—and other hormonal single or combined formulations (natural or synthetic, in various form of drug delivery—pills, injections, patches, gel, implants,etc.) will allow to reduce duration and frequency of ultrasound pulses, therefore reduce tissue exposure to ultrasound irradiation and to obtain an effective and reliable contraception with reduced risks and side effects. Combination of the device and drugs will also allow to substantially reduce effective drug doses and therefore reduce drug induced side and negative health effects to negligible levels. Also it may reduce device power consumption and extend battery life.

Testosterone may be in a form of testosterone enanthate (TE), 19-nortestosterone hexoxyphenylpropionate (19NT), and testosterone buciclate (TB), 7-alpha-methyl-nortestosterone (MENT), Testosterone undecanoate (TU).

Doses may be used as followed: weekly injections up to 200 mg TE may be administered; up to 1000-mg implants, every 4 or 6 months may be used; up to 300 mg depot medroxyprogesterone acetate, im, every 3 months may be administered; maximal testosterone depot dose (1200 mg) may be used; up to 300-mg dose of depot medroxyprogesterone acetate (DMPA)-progestin addition to testosterone. Pills −25 mg or less testosterone propionate (TP) daily may be administered. Pellets consist of fused crystalline testosterone, which is usually inserted subcutaneous using a trocar, with the patient under local anesthesia, into the lower abdominal wall at a dose up to 800 mg (4×200 mg pellets, each releasing 1.3 mg/day or less may be used. 1000 mg or less TU administered every 6 wk may be used. DSG+TE−300 mg+100 mg or less may be used.

combination of DMPA with testosterone—four 200-mg T pellets plus a single intramuscular injection of 300 mg depot medroxyprogesterone acetate (DMPA) or less may be used.

Cyproterone acetate (CPA)−200 mg/d or less; combination of CPA in doses of 25-100 mg/d with 100 mg TE per week or less; 500 μg LNG orally+TE (100 mg/wk) or less; 300 μg or less oral desogestrel daily in combination with 50 mg or less im TE weekly may be used.

Non-hormonal chemicals which suppress spermatogenesis and motility such as gossypol and triptolide may be used in combination with the device.

Motility-reducing chemical agents based on Trypterigium wilfordii, Ornidazole and others. Glyceraldehyde-3-phosphate dehydrogenase is inhibited limiting ATP production from glucose, thus reducing sperm motility and may be used in combination with the device.

CatSper targeting agents and compounds may be used in combination with the device.

Combination of the device with heat methods of reducing male fertility may be used.

A combination with immunization methods—antisperm antibodies may contribute to subfertility and infertility in both men and women. Suppressing spermatogenesis to low base level in combination with ultrasound device may be very effective and reliable contraceptive method.

Example of implementation: The vas deferens OD is 3 mm, lumen ID is 0.25 mm. For in vitro tests a vas deferens was simulated by a silicone tube with the same dimensions as the vas deferens. The sperm from a fertile volunteer was placed in the internal volume of the lumen. A microscope was used to estimate motility of the spermatozoa The device is made as a hollow cylinder with ID=4 mm, OD=10 mm, and length 10 mm. The device is made of two half cylinders. The upper half is made of piezoelectric ceramic with metalized internal and external surfaces that are used for contacts. The device is placed concentrically to the vas deferens. An ultrasound transmitting silicone gel is used to fill voids between the vas deferens and the internal surface of the device. The electrical pulses with frequency 200 kHz from a control circuitry are applied to the contacts that cause contraction of the piezoceramic and generation of ultrasound waves directed toward the axis of vas deferens and having a focus point there. The power is 0.06 W.

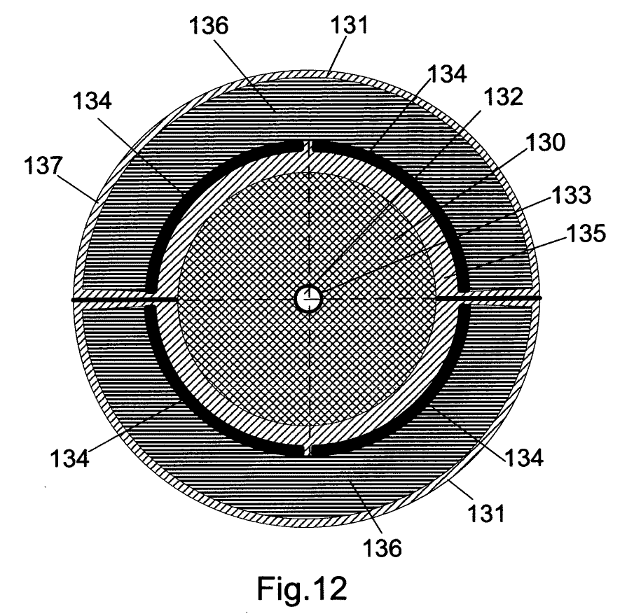

FIG. 12 shows the cross-section of the device per another embodiment. The cross-section shown is made perpendicular to the axis of vas deferens 130. The device is comprised of two or more parts 131 symmetrical relative the lumen 132 axis 133. Every device part consists of one or more ultrasound emitter 134 positioned predominantly symmetric relative the lumen axis 133. The emitter may be flat or concave and directed toward the lumen 132. An ultrasound coupling medium is placed between the emitters 134 and vas deferens 130. The device is provided at least one battery 136 and controller (not shown). All components of the device are enclosed in biocompatible housing 137.

The emitters 134 operate simultaneously or in sequence that allows irradiation of the lumen 132 from different angles.

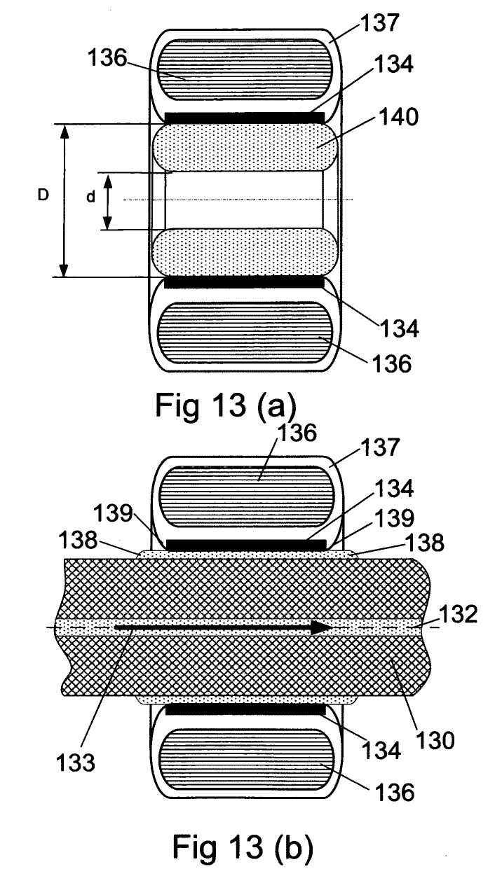

Human vas deference has diameter variations from 2 to 4 millimeters. Another embodiment of the device shown in FIGS. 13 (a,b) is provided with an elastic element 140 that is coaxial to the emitters 134. The internal diameter d of the elastic element 140 (FIG. 13(a)) is made less than the distance D between the opposing emitters 134 or the internal diameter of the device.

The internal diameter of the device or the distance D between opposing emitters 134 is made more than the largest expected external diameter of the vas deferens. For human D>4 mm. The internal diameter d of the elastic element 140 is made less than the expected minimal external diameter on the vas deferens. For human D<2 mm.

The elastic element is made of a synthetic or natural polymer or rubber with acoustic properties and density close to the acoustic properties and density of the smooth muscle of the vas deferens. The material of the elastic element 140 is softer than the smooth muscle of the vas deference and biocompatible. The elastic element 140 may be made of silicone rubber.

After the device is positioned on the vas deferens 132 the elastic element 140 is compressed along its radius and expands along its axis. The elastic element fills the gap between the external surface of the vas deferens 132 and emitters 134 providing acoustic coupling. Expanded parts 138 of the elastic element 140 provide reduction of localized stress on the vas deference by the device edges 139.

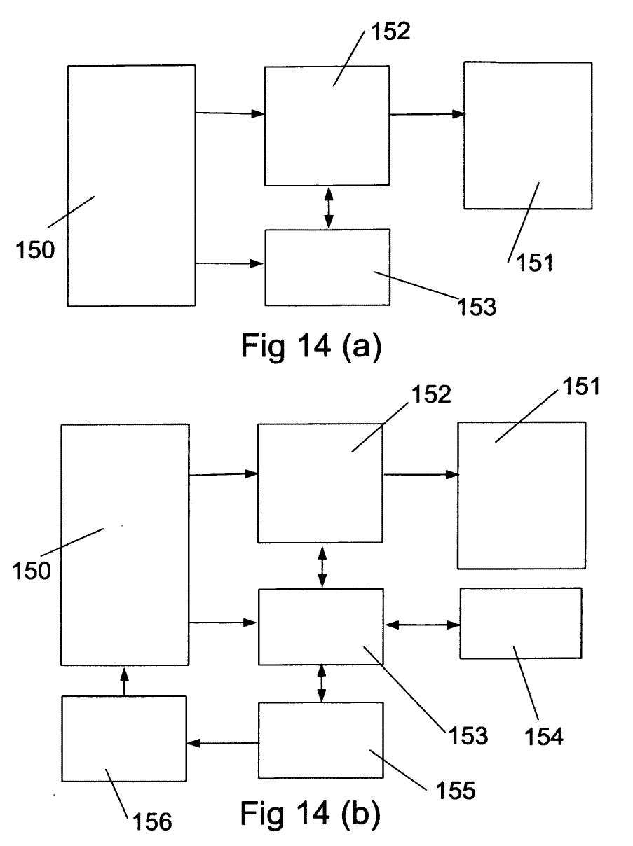

FIG. 14(a,b) shows a block diagram of an electrical and control schematic for the device per current invention.

In one embodiment (FIG. 14 (a)) the device consists of a battery 150, energy emitter 151, driver circuitry 152 and controller 153. The driver circuitry 152 matched voltage, frequency and power provided by the battery 150 to the need of the emitter 151. The controller 153 provider timing and operation sequencing for the driver 152. The controller 153 and driver 152 may be made as solid state circuitry on one or several semiconductor chips.

In other embodiment (FIG. 14(b)) the device is additionally provided with an antenna 155 that allows RF communication with a remote controlling device for remote turn on/of the device or reporting the device and/or battery status.

In other embodiment the battery 150 is a rechargeable battery and may be recharged by an internal charger 156. The internal charger 156 may operate on conversion of mechanical (vibration) or thermal or magnetic or chemical energy to electricity for restoring battery 150 charge. In combination with the antenna 155, the charger 156 can be used for RF recharging of the battery 150.

In another embodiment the device is provided with a sensor 154 that is capable to detect an increase in peristaltic activity of the vas deferens in comparison with slower peristaltic activity during sexual rest. The example of the sensor may be a piezosensor that detects periodic wall pressure changes due to contraction of the vas deferens. The controller 153 analyses the frequency of the peristaltic activity and correspondingly adjusts power or frequency or duty cycle of emitter 151 operation. Although the present invention has been particularly described with reference to embodiments thereof, it should be readily apparent to those of ordinary skill in the art that various changes, modifications and substitutes are intended within the form and details thereof, without departing from the spirit and scope of the invention. Accordingly, it will be appreciated that in numerous instances some features of the invention will be employed without a corresponding use of other features. Further, those skilled in the art will understand that variations can be made in the number and arrangement of components illustrated in the above figures. It is intended that the scope of the appended claims include such changes and modifications.

1. A method of contraception for use with reproductive cells that pass through a tube wall of a tube in a body comprising the step of applying energy through the tube wall to some of the reproductive cells that pass through the tube to reduce fertilization ability of the some of the reproductive cells.

2. The method of claim 1 wherein the reproductive cells are sperm, the tube is a vas deferens through which the sperm travel, and wherein the step of applying energy applies pressure pulses to the vas deferens to reduce fertilization ability of at least some of the sperm that travel through the vas deferens.

3. The method of claim 2, wherein the step of applying the pressure pulses includes the step of forming high pressure pulses in a lumen of the vas deferens and low pressure pulses in a smooth muscle of the vas deferens.

4. The method of claim 3 wherein the step of applying pressure pulses includes formation of acoustic waves outside the vas deferens and directing the acoustic waves toward a center of the vas deferens lumen to create the high pressure pulses in the lumen.

5. The method of claim 4 wherein multiple acoustic waves are formed around the vas deferens, the acoustic waves are directed predominantly toward the center of the vas deferens lumen, and at least two acoustic waves intersect at substantially a same point of the vas deferens lumen.

6. The method of claim 2 wherein the step of applying pressure pulses is performed using an acoustic wave emitter that is permanently positioned relative to the vas deferens.

7. The method of claim 6 wherein the acoustic wave emitter is permanently implanted in the scrotum.

8. An apparatus for use with reproductive cells that pass through a tube in a body comprising:
an energy transmitter that delivers energy to the reproductive cells that pass through the tube in the body.

9. The device of claim 8 wherein the reproductive cells are sperm, the tube is a vas deferens, and the body is a male mammal, such that sperm travel through the vas deferens, and
wherein the energy transmitter is an acoustic wave emitter that delivers the energy as an emitted acoustic wave to reduce fertilization ability of at least some of the sperm that travel through the vas deferens.

10. The device of claim 9 further including:
control circuitry that controls the acoustic wave emitter;
a power source that provides electrical power to the control circuitry and the acoustic wave emitter; and
an attachment mechanism for attachment of the acoustic wave emitter relative to the vas deferens.

11. The device of claim 10 further including more than one acoustic wave emitter, the emitters being placed so that emitting surfaces of the emitters are disposed tangential to a common arc that has a with the radius larger than a radius of the vas deferens, wherein the emitters are oriented so that the emitting surfaces are facing the center of the common arc.

12. The device of claim 11 wherein the acoustic wave emitter is a donut shaped segment of piezoelectric material, and an internal radius of the segment is made larger than a radius of the vas deferens, wherein an internal surface of the segment emits the acoustic waves.

13. The device of claim 12 further including more than one donut shape segment, wherein the emitting surfaces of the emitters are oriented concentrically.

14. The device of claim 10 further including an acoustic wave reflector mounted to face the acoustic wave emitter, a distance between the acoustic wave emitter and the reflector being larger than a diameter of the vas deferens.

15. The device of claim 10 further including at least one sensor that detects a peristaltic contraction of the vas deferens.

16. The device of claims 10 further including an antenna for communicating signals with the control circuitry.

17. The device of claim 10 further including a light source.

18. The device of claim 10 further including electrodes that form an electrical field that is applied to an internal volume of the vas deferens wherein the electrodes are electrically isolated to minimize electrical current.

19. The device of claim 10 further including an actuator for temporal pinching of the vas deferens.

20. The device of claim 10 further comprising a mechanism that reduces the vas deference cross-section size and/or shape without lumen occlusion.
